SEQUENCE LISTING (4 sequences)

SEQ ID NO: 1 - protein, 9 aa, Pseudonocardia thermophila
IHMPDPDAV

SEQ ID NO: 2 - protein, 14 aa, Pseudonocardia thermophila
DGLPVGLMIVGKHF

SEQ ID NO: 3 - protein, 509 aa, Pseudonocardia thermophila
MIHMPDPDAVARMAAEGRFGIPVVVICRTYHDLGHRPARLVEQGRRAVRGGGPHPAAALVDPPRRRGRQARRVGGADLDHRNLGGAARRPHGRGQDNVAVAGVPMMNGSRTLEGFVPAEDATVVSRLLAAGATIAGKSVCEDLCFSGGSHTSKPGTVHNPWDMSRSAGGSSSGSGALVAAGEVDMAIGGDQGGSIRIPSAYCGTVGHKPTHGLVPYTGGFPIEQSIDHLGPITRTVADAALMLSVIAGRDGLDPRQPDVVEVQDYVGALAESVSGLRIGVLQEGFGHPNSEPEVDDTVRAAVGTLREAGFTVEDVSVPWHLHATAIWDVLATEGGLWQMVEGNAYGMHWKGHYDPSLIAYYGRKWREDPAQFSETVKLVALAGRYALTTQYGRHYAMARNLAPKLVAAYDAALSNYDVLVMPTLPMRATVLPGPDAPVEEILARGLEMLANTAPFDVTGHPACSVPAGLADGLPVGLMIVGKHFDDATVLKVANAFEQAVGGFPTPAAZ

SEQ ID NO: 4 - DNA, 1527 bp, Pseudonocardia thermophila
atgatccacatgccggacccggacgcggtggcccggatggccgcagagggtcggttcgggatccccgtagtagtgatctgccgtacctaccacgacctcggtcaccggcctgctcggctcgtggaacaaggtcgaagagctgtacgcggaggtggcccccaccccgccgcagcgctcgtggacccgccccgacgccgaggacgacaagctcggcgcgtgggcggtgcagacctcgatcaccgaaacctcggaggggccgctcgccggccgcacggtcgcggtcaggacaacgtcgccgtcgccggcgtgccgatgatgaacggatcgcggaccctggagggcttcgtccccgccgaggacgccaccgtcgtgtcccggctgctcgccgccggcgcgaccatcgccggcaagtcggtgtgcgaggacctgtgcttctccggcggctcgcacaccagcaagccgggaaccgtgcacaacccgtgggacatgagccgctcggcgggtggttcctcgagcggtagcggggcgctggtggccgccggcgaggtcgacatggccatcggcggtgaccagggcgggtcgatccgcatcccgtcggcgtactgcggcaccgtcgggcacaagccgacccacggcctggtgccgtacaccggaggcttcccgatcgagcagtcgatcgaccacctcggcccgatcacccgcaccgtcgccgacgccgcgctcatgctgtcggtgatcgccggccgcgacgggctcgacccgcgccagcccgacgtcgtcgaggtgcaggactacgtcggcgcgctggccgagtcggtgagcgggctgcgcatcggcgtcctgcaggagggcttcggtcaccccaactccgagcccgaggtcgacgacaccgtgcgcgctgctgtcgggacactgcgcgaggccggcttcaccgtcgaggacgtctcggtcccgtggcacctgcacgccacggccatctgggacgtgctcgccaccgagggcggcctgtggcagatggtcgagggcaacgcctacggcatgcactggaagggccactacgacccgtcgctcatcgcctactacgggcgcaagtggcgggaggacccggcgcagttctccgagacggtcaagctcgtcgccctcgccggccgctacgcgctcacgacccagtacgggcgccactacgcgatggcgcgcaacctcgccccgaagctggtcgcggcctacgacgcggcgctgtcgaactacgacgtgctcgtcatgccgacgctgccgatgcgcgccaccgtgctgcccggcccggacgcgcccgtcgaggagatcctcgccaggggcctggagatgctggccaacaccgcaccgttcgacgtcaccgggcacccggcctgctctgtcccggccggcctcgccgacggtctcccggtcggcctgatgatcgtcggcaagcacttcgacgacgccaccgtgctgaaggtggcgaacgccttcgaacaggccgtcggcggcttcccgacccccgctgcctga

(19) United States
(12) Patent Application Publication
(10) Pub. No.: US 2007/0172933 A1
(43) Pub. Date: Jul. 26, 2007

(54) Thermally stable amidases

(76) Inventors: Ksenia Egorova, Hamburg (DE); Garabed Antranikian, Hittfeld-Waldesruhd (DE); Harald Trauthwein, Munchen (DE); Stefan Verseck, Hanau (DE); Uwe Dingerdissen, Seeheim-Jugenheim (DE)

Correspondence Address: CONNOLLY BOVE LODGE & HUTZ, LLP, P O BOX 2207, WILMINGTON, DE, 19899, US

(21) Appl. No.: 10/549,782

(22) PCT Filed: Feb. 16, 2004
(86) PCT No.: PCT/EP04/01430

§ 371 Date: Oct. 2, 2005

(30) Foreign Application Priority Data
Mar. 21, 2003 (DE) 103 12 842.5

(51) Int. Cl. C12P 13/00; C07H 21/04; C12N 9/80; C12N 15/74; C12N 1/21

(52) U.S. Cl. 435/128; 435/228; 435/252.3; 435/471; 536/23.2

(57) ABSTRACT

The invention relates to novel amidases which can be isolated from thermophilic bacteria, in particular from thermophilic Actinomycetes, e.g. Pseudonocardia thermophila, in which the resultant amidases not only have a high temperature stability, but are also distinguished by the enantioselective reaction of a broad substrate spectrum.

### DESCRIPTION

The invention relates to novel amidases which can be isolated from thermophilic bacteria, in particular from thermophilic Actinomycetes, e.g. Pseudonocardia thermophila, in which the resultant amidases not only have a high temperature stability, but are also distinguished by the enantioselective reaction of a broad substrate spectrum.

Enzymatic methods are increasingly being incorporated into organic synthesis, in particular when carrying out enantioselective syntheses, e.g. in the production of optically pure pharmaceutical active compounds, amino acids, acrylic acids or hydroxamic acids. In addition, amidases participate in the breakdown of xenobiotic compounds.

Amidases catalyze the reaction of amides to form their conjugate carboxylic acids and amines or to form ammonia. In the interim, a number of bacterial amidases have been identified, including also some amidases from mesophilic Actinomycetes (Bhalla, T.C., et al.; Science letters 11-12; 139-141, 1997; Hirrlinger, B.; et al; J. Bacteriol. 178, 3501-3507, 1996; Kobayashi, M., et al.; Eur. J. Biochem., 217, 327-336, 1993; Kotlova, E.K.; et al.; Biochemistry (Mosc.) 64, 384-389, 1999; Mayaux, J.F. et al.; J. Bacteriol. 173, 6694-6704, 1991; Mayaux, J.F.; J. Bacteriol. 172, 6764-6773, 1990; Nawaz, M.S., et al.; Appl. Environ. Microbiol., 60, 3343-3348, 1994), no thermostable amidase from thermophilic Actinomycetes having been described, however.

Thermoactive amidases, in contrast, have hitherto only been found in Klebsiella pneumoniae NCTR 1 (Nawaz, M.S.; J. Bacteriol. 178, 2397-2401, 1996) and in Sulfolobus solfataricus (d' Abusco, A.S.; Extremophiles, 5, 183-192, 2001). Precisely the preparation of further amidases, in particular thermostable amidases, would, however, be of great technical interest, since, using such enzymes, a relatively broad spectrum opens up of enzymatic reactions which can be carried out industrially. The object underlying the present invention was therefore to provide novel thermally stable amidases.

The object is achieved by amidases which contain an N-terminal sequence (SEQ ID No. 1)

IHMPDPDAV (SEQ ID No. 1)

and/or an amino acid sequence

DGLPVGLMIVGKHF (SEQ ID No. 2)

or a sequence having a homology of greater than 50%, preferably greater than 70%, particularly preferably greater than 80%, with SEQ ID No. 1 and/or SEQ ID No. 2.

The novel amidases are obtainable, e.g. from thermophilic bacteria, in particular from thermophilic Actinomycetes, e.g. from Pseudonocardia thermophila. For instance, a constitutively expressed amidase in Pseudonocardia thermophila having a molecular weight between 47 and 53 kDa can be isolated, which preferentially occurs as dimer having a molecular weight between 100 and 140 kDa. Identification of further mutant or allelic variants of amidases can be performed e.g. on the basis of nucleic acid probes which are complementary to a DNA sequence coding for the amino acid sequence SEQ ID No. 1 or SEQ ID No. 2. The hybridization of such a probe is performed under stringent conditions, e.g. at 60° C., 0.1×SSC, 0.1%

Although the native amidases found can be identified by the N-terminal sequence SEQ ID No. 1 and SEQ ID No. 2, or a homologous variant thereof, for amidase activity, at least the N-terminal sequence is not absolutely required. Therefore, the present invention also comprises those above-described amidases the N-terminal end of which is artificially deleted. Likewise, the partial sequence SEQ ID No. 2 can also be artificially deleted. Furthermore, the present invention comprises amidases having an amino acid sequence according to SEQ ID No. 3, or an amino acid sequence having a homology of at least 50% therewith, preferably having a homology of over 70%, in particular over 80%, therewith.

The present invention further relates to nucleic acids coding for an inventive amidase having a sequence according to SEQ ID No. 4, or having a nucleotide sequence having a homology of over 60%, preferably over 75%, in particular preferably over 90%, therewith. SEQ ID No. 4 codes for an amidase having an amino acid sequence SEQ ID No. 3.

The amidases described can be purified, as described hereinafter, from a cell-free crude extract of thermophilic bacteria which is obtainable, e.g., by ultrasonic disruption of Pseudonocardia thermophila cells in a phosphate buffer:
a) centrifugation of the crude extract at 10 000 to 20 000 rpm and subsequent addition of a 1 M salt solution, preferably a KCl solution,
b) chromatographic separation of the supernatant on a hydrophobic column using a reverse gradient of a salt solution, preferably a KCl solution, from 1 M to 0 M,
c) ultrafiltration of the fraction showing amidase activity obtained from b) on a 10 kDa cut-off membrane,
d) ion-exchange chromatography of the protein fraction obtained from c) using a gradient from 0 M to 0.5 M of a salt solution, preferably an NaCl solution
e) chromatography of the fraction showing amidase activity obtained from d) using a 0.1% strength salt solution, preferably a 150 mM NaCl solution, and desalting the purified amidase fraction.

Preferably, use is made of alkali metal halide salt solutions, e.g. NaCl or KCl solutions, for carrying out the individual chromatographic purification steps. The chromatographic separations are preferably carried out between pH 6.5 and 8.0, in which case the pH can be set, e.g. by using a standard phosphate buffer (pH 7.2). The ion-exchange chromatography is preferably carried out at a pH between 7.5 and 8.5.

The amidase activity of the resultant fractions can be determined using a hydrolysis test, with benzamide being able to be used as substrate. The products produced enzymatically, benzoic acid and ammonia, can be determined by HPLC (benzoic acid) or the phenyl hydrochloride method (detection of ammonium ions).

The inventive amidases thus produced are distinguished by a high temperature stability. The specific activity of the enzyme is not significantly impaired until at temperatures around 80° C. at relatively short reaction times of more than one hour. In addition, the amidases described have a temperature optimum between 50 and 750C. The amidases described, furthermore, exhibit a significant activity between 30 and 850C., but preferably, the enzymes are usable at reaction temperatures between 60 and 70° C.

Furthermore, the specific activity of the inventive amidases is also retained over a broad pH range. For instance, the amidases have a significant enzymatic activity between pH 3.5 and pH 11.5, the specific activity scarcely decreasing from the optimum between pH 6.0 and 7.5 over the range of pH 4.5 and 10.0.

The high temperature optimum, the good thermal stability and the wide pH range in which the enzymes are active open up an interesting scope for optimizing amidase-catalyzed processes, such as the hydrolysis of amides.

In addition, it can be shown that the amidases described here are not sensitive to a large number of reagents and ions. For instance, the amidases are insensitive, e.g. to chelating agents, e.g. EDTA, or to detergents such as SDS or Triton. Also, the dependence on ionic cofactors does not appear to be a factor. By adding DTT, the enzymatic activity of the amidases can even be increased.

The inventive amidases produced and characterized differ in their properties significantly from the microbial amidases known hitherto, as can be seen from the exemplary compilation in Tab. 4. It is noteworthy, in particular, here that the enzyme isolated from Pseudonocardia thermophila is the first known naturally occurring homotrimeric amidase.

TABLE 4

| | Characteristics | | | | | | | |
|---|---|---|---|---|---|---|---|---|
| | | Molar | Subunits | | | IEF | Metal | Substrate |
| | | mass | kDa | pH | T opt. | point | co- | specificity |
| Microorganism | Expression | in kDa | (number) | opt. | in ° C. | pH | factors | (amides) |
| Pseudonocardia thermophila | constitutive | 110 | 50(2) | 7.0 | 70 | 4.2 | n.d. | Al, Ar, Cy, As |
| Agrobacterium tumefaciens d3^{a)} | inducible | 490 | 63(8) | 7.5 | 22 | n.d. | n.d. | Al, Ar |
| Bacillus stearothermophilus^{b)} | inducible | 39 | monomer | 7.0 | 55 | n.d. | n.d. | Al |
| Brevibacterium sp. R312^{c)} | inducible | 110 | 43(2) | 7.5 | 30 | n.d. | n.d. | arylpropioamide |
| Comamonas acidovorans KPO^{d)} | constitutive | 54 | monomer | 8.0 | 30 | n.d. | n.d. | ketoprofen |
| DSM 6320^{e)} | inducible | 125 | 66(2) | 8.5 | 40 | 4.2 | n.d. | L-carnitine |
| Helicobacter pylori^{f)} | constitutive | 40 | monomer | 7.0 | 55 | n.d. | n.d. | Al |
| Klebsiella pneumoniae NCTR1^{g)} | inducible | 62 | monomer | 7.0 | 65 | n.d. | Co&Fe | Al |
| Mycobacterium neoaurum^{h)} | constitutive | 136 | 40(4) | 8.0 | 50 | 4.2 | n.d. | Al, As |
| Mycobacterium smegmatisⁱ⁾ | constitutive | 50 | monomer | 7.5 | 22 | n.d. | n.d. | Cy |
| Ochrobactrum antropi SV3^{j)} | inducible | 40 | 63(8) | 9.0 | 22 | n.d. | n.d. | As |
| Pseudomonas aeruginosa^{k)} | inducible | 200 | monomer | 7.0 | 55 | n.d. | n.d. | Al |
| Pseudomonas chlororaphis B23^{l)} | inducible | 105 | 54(2) | 7.0 | 50 | n.d. | no metal | Al, Ar, Cy, As |
| Rhodococcus rhodochrous J1^{m)} | inducible | 110 | 55(2) | 7.9 | 55 | n.d. | n.d. | Al, Ar, Cy, As |
| Rhodococcus rhodochrous M8ⁿ⁾ | constitutive | 150 | 42(4) | 7.0 | 55 | n.d. | n.d. | Al |
| Rhodococcus erythropolis MP50^{o)} | inducible | 480 | 61(8) | 7.5 | 55 | n.d. | n.d. | Al, Ar, Cy |
| Rhodococcus sp.^{p)} | constitutive | 360 | 44.5(8) | 8.5 | 40 | 4.0 | Fe | Al |
| Rhodococcus sp. NHB-2^{q)} | inducible | n.d. | n.d. | 8.0 | 55 | n.d. | n.d. | Al |
| Rhodococcus sp.^{r)} | inducible | 118 | 48.5(2) | 7.5 | 30 | n.d. | n.d. | arylpropionamide |
| Stenotrophomonas maltophila^{s)} | inducible | 38 | monomer | 6.0 | 40 | 5.8 | n.d. | As, peptides |
| Sulfolobus solfataricus^{t)} | inducible | 56 | monomer | 7.5 | 95 | 5.94 | n.d. | Al, Ar, Cy |

Al = aliphatic amides,
Ar = aromatic amides,
Cy = cyclic amides,
As = amino acid amides,
n.d. = not determined
^{a)}Trott et al.; Mikrobiology 147, 1815-1824, 2001;
^{b)}Cheong, et al., Enzyme Microb. Technol. 26, 152-158, 2000;
^{c)}Mayaux et al., J. Bacteriol. 172, 6764-6773, 1990;
^{d)}Yamamoto et al., Appl. & Environ. Microbiol. 1, 152-155, 1996;
^{e)}Joeres et al., Appl. Microbiol. Biotechnol. 40, 606-610, 1994,
^{f)}Skouloubris, et al., Mol. Microbiol. 25, 989-998, 1997;
^{g)}Nawaz et al., J. Bacteriol. 178, 2397-2401, 1996;
^{h)}Hermes et al., Appl. & Environ. Microbiol. 1, 153-159, 1994;
ⁱ⁾Boshoff et al., J. Bacteriol. 180, 5809-5814, 1998;
^{j)}Komeda et al., Eur. J. Biochem. 267, 2028-2035, 2000;
^{k)}Nawaz et al., Appl. Biochem. & Biotechnol. 28/29, 865-875, 1991;
^{l)}Ciskanik et al., Appl. Environ. Microbiol. 61, 998-1003, 1995,
^{m)}Kobayashi et al., Eur. J. Biochem., 217, 327-336, 1993;
ⁿ⁾Kotlova et al., Biochemistry (Mosc), 64, 384-389, 1999;
^{o)}Hirrlinger et al., J. Bacteriol. 178, 3501-3507, 1996;
^{p)}Nawaz et al., Appl. Environ. Microbiol. 60, 3343-3348, 1994;
^{q)}Bhalla et al., Science letters, 11-12, 139-142, 1997;
^{r)}Mayaux et al., J. Bacteriol., 173, 6694-6704, 1991;
^{s)}Neumann et al., Appl. Microbiol. & Biotechnol. 58, 773-780, 2002;
^{t)}d'Abusco et al., Extremophiles, 5, 183-192, 2001

A great advantage of the novel amidases is their broad substrate spectrum. For instance, using the enzymes, not only aliphatic, aromatic, cyclic, heterocyclic but also amino acid amides may be hydrolyzed. In particular aliphatic amides having one to ten carbon atoms, aromatic amides having 5 to 12 carbon atoms, heterocyclic amines having 4 to 10 carbon atoms and having one to four hetero atoms, e.g. selected from the group N, S, O, P or L-amino acid amides can be reacted.

The amidases described are distinguished by, in addition to the broad substrate spectrum, also by a high enantioselectivity. In particular, the production of S enantiomers, e.g. of (hetero)cyclic acids and, in particular, aromatic, aliphatic or aromatic-aliphatic acids, is preferred.

### BRIEF DESCRIPTION OF THE FIGURES

FIG. 1 shows the results of an SDS-PAGE electrophoresis using different enzyme samples which were taken during the purification (tracks 2 to 5). Tracks 6 and 7 show a zymogram of the crude extract and the purified sample. Track 1 is a molecular weight standard. FIG. 2 shows the result of the molecular weight determination of the native amidase using gel filtration. FIG. 3 shows the specific activity of an inventive purified amidase as a function of reaction temperature, FIG. 4 as a function of the pH. FIG. 5 shows the stability of the purified amidase at different temperatures. In FIG. 6, the acetyl-transferase activity based on example substrates is shown. In FIG. 7, the enantioselective enzymatic reaction is shown using 2-phenylpropionamide as substrate.

Hereinafter, some example embodiments are given which, however, are not to be understood as limiting.

### EXAMPLE 1

Culture of Pseudonocardia Thermophila (DSMZ 43832)

The Pseudonocardia thermophila strain used was acquired from the German collection of microorganisms and cell cultures (DSMZ, Brunswick, Germany). The cells were cultured, where not stated otherwise, as described in Yamaki, T.; et al.; J. Ferment. Bioeng., 83, 474-477, 1997. The medium (pH 7.2) comprises per liter: 5 g of yeast extract, 4 g of soluble starch, 0.3 g of KH2PO4, 0.6 g of Na₂HPO₄, 0.1 g of MgSO₄.7H₂O, 5 g of NaCl. The cells were harvested after 3 days culturing in a shaking flask at 50° C., 150 rpm. The yield was 10 g of cells (wet weight) per liter of medium.

### EXAMPLE 2:

Purification of the Amidase:

The amidase was purified at room temperature. 7 g of cell material (wet weight) were washed for this 2 times using 60 mM K-Na phosphate buffer (standard buffer), pH 7.2, resuspended in 50 ml of the buffer and disrupted by means of ultrasound. After 20-minute centrifugation at 13 000 rpm, a 1 M KCl solution was added to the cell-free extract. The supernatant (50 ml) was placed on a hydrophobic Phenyl-Sepharose Fast Flow column (Pharmacia, Sweden) which had been equilibrated with a 1 M KC1 standard buffer. The protein was then eluted using a linear reverse gradient of 1 M KC1 to 0 M KC1 solution. The amidase-containing fractions were combined and dialyzed against 2 l of standard buffer (pH 8.0). The resultant protein fraction was then concentrated 12-fold and ultrafiltered through a 10 kDa cut-off membrane (Amicon). The sample was then further purified via ion-exchange chromatography. For this, a UNO-Q12 column (BioRad) was used, with the purified protein being purified using a linear NaCl gradient (0 to 0.5 M NaCl in standard buffer, pH 8.0). The enzymatically active fractions were combined, concentrated 2-fold and fractionated using a preparative HiLoad 26/60 Superdex 200 column (Pharmacia, Sweden) using standard buffer, pH 7.2, containing 150 mM NaCl. The protein fraction having amidase activity was collected and desalted via a Sephadex PD-10 column (Pharmacia, Sweden). The protein concentration was determined using bovine serum albumin as standard as described in Bradford, M. M.; Anal. Biochem. 71, 248-254, 1976.

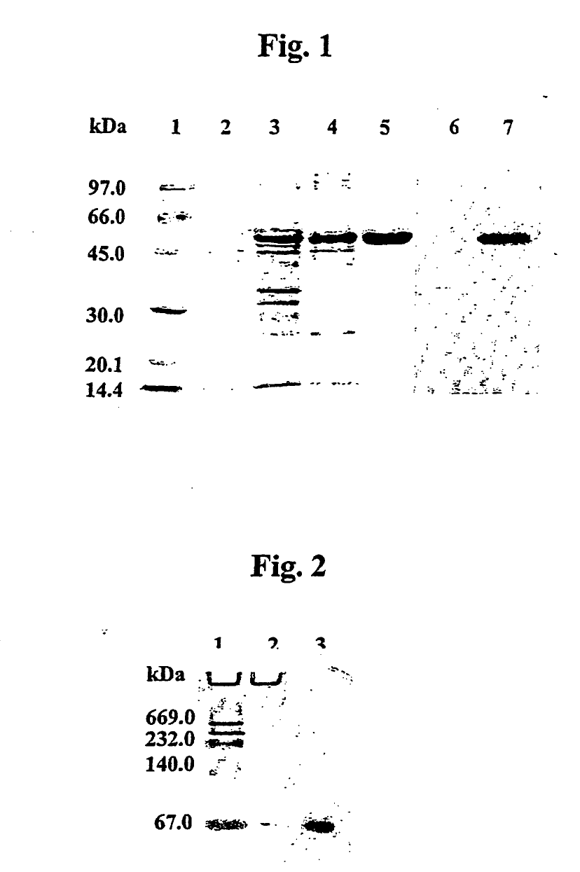

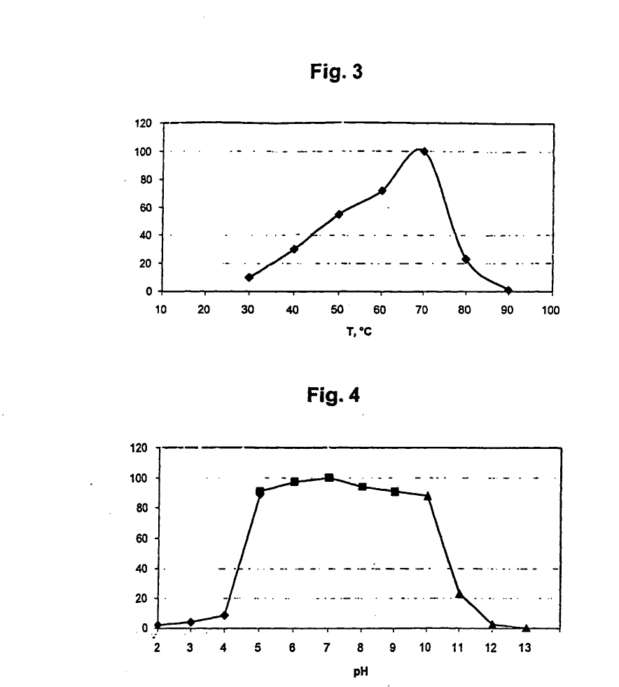

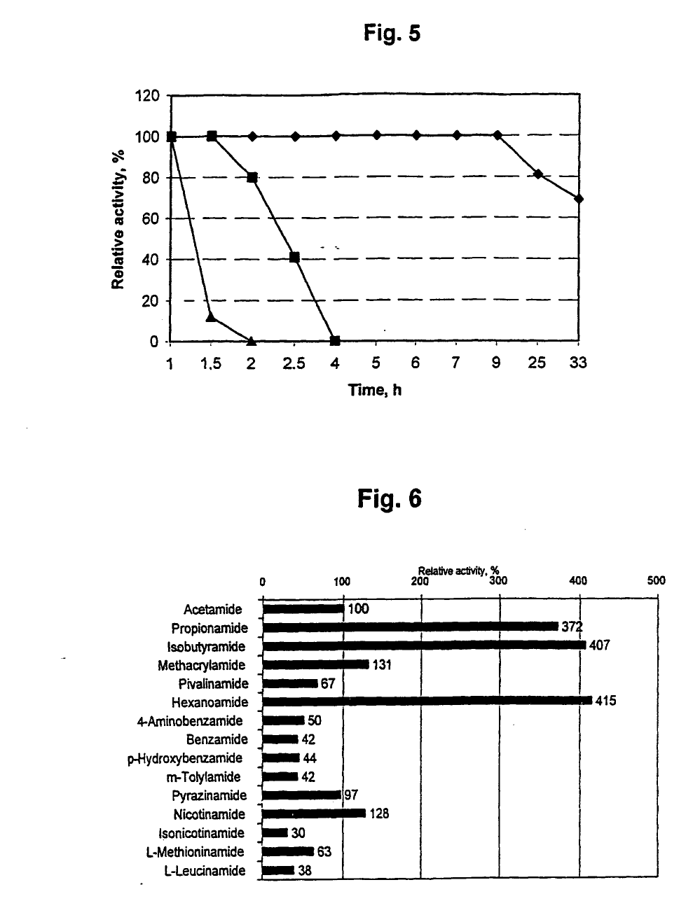

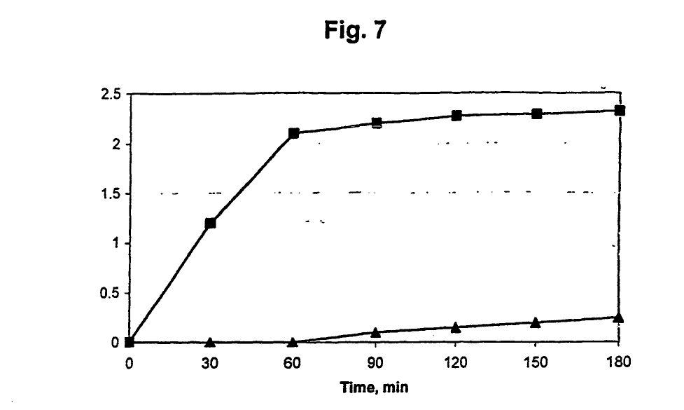

The result of the purification is shown in FIG. 1 and Table 1.

The success of purification was studied using sodium dodecyl sulfate-polyacrylamide (SDS-PAGE) gel electrophoresis. For this, a ready-prepared gel (Novex, Invitrogen, Netherlands) was used with a tris-glycine gradient of 4-20%. As standard proteins, use was made of phosphorylase b (94 kDa), bovine albumin (67 kDa), chicken ovalbumin (43 kDa), carbonic anhydrase (30 kDa), soybean trypsin inhibitor (20.1 kDa) and bovine a-lactalbumin (14.4 kDa) (Pharmacia, Sweden). The protein bands were stained using Coomassie Blue R-250.

To verify the amidase activity on the gel, the SDS gel was washed for 60 minutes in 2.5% Triton X-100 and then incubated for 30 minutes at 60° C. in K-Na phosphate buffer (pH 7.0) containing 70 mM propionamide and 0.7 M hydroxylamine hydrochloride, the pH having been set to 7.0 using 10 M NaOH. The gel was thereafter washed in water for 2-3 sec. and covered with an acidic iron chloride solution (0.1 M FeCl₃ in 0.5 M HCl)

The iron applied reacts with the enzymatically formed hydroxamic acid, forming a reddish-brown band. The intensity of staining corresponds to the enzymatic activity of the amidase. The activity test just described is also termed zymogram hereinafter in the description.

FIG. 1 shows the results of an SDS-PAGE electrophoresis using different samples (in each case 5 gg of protein), from the fractions obtained during the purification. The proteins were stained using Coomassie Blue. Track 1 shows the standard proteins, track 2 the crude extract, track 3 a sample after purification on the Phenyl-Sepharose column, track 4 a sample after ion-exchange chromatography on a UNO Q12 column, track 5 a sample after purification on a Superdex 200 column. Tracks 6 and 7 show the zymogram of the crude extract and the purified sample.

TABLE 1
Results of purification of the amidase
from Pseudonocardia thermophila

| | Protein | Total | Specific | | Degree of |
|---|---|---|---|---|---|
| Purification | in mg | activity | activity | Yield | purifi- |
| step | (total) | (U)^{a} | (U) | (U × mg⁻¹) | cation |
| Crude | 85 | 34.7 | 0.4 | 100 | 1-fold |
| extract^{b} | | | | | |
| Phenyl- | 4.4 | 25.2 | 5.7 | 72.6 | 14-fold |
| Sepharose | | | | | |
| UNO Q12 | 2.0 | 16.8 | 8.5 | 48.3 | 21-fold |
| Superdex 200 | 0.46 | 9.1 | 19.5 | 26.2 | 48-fold |

^{a}one U of the amidase catalyzes the formation of 1 μmol of benzoic acid per minute and per mg of protein under standard conditions
^{b}after culture at 50° C., centrifugation of the 1-liter culture (7 g of cell material (wet weight)), washing two times with standard buffer (pH 7.2) and ultrasound disruption.

Using the purification method described, the enzymatically active amidase was obtained with a yield of 26.2%. The specific activity was 19.5 U/mg of protein, based on benzamide as substrate. The amidase activity was confirmed by HPLC analysis of the products of the enzyatically converted benzamide.

### EXAMPLE 3

Determination of the Molecular Weight of the Amidase

The molecular weight of the amidase was determined by means of gel filtration on a Superdex 200 column (Amersham) using a 60 mM K-Na phosphate buffer (pH 7.2) containing 150 mM NaCl. The column was calibrated using Blue Dextran (2000 kDa), sweet potato β-amylase (200 kDa), yeast alcohol dehydrogenase (150 kDa), bovine serum albumin (66 kDa), carbonic anhydrase (29 kDa) and cytochrome c (12.4 kDa) (Sigma Aldrich, Germany).

The molecular weight of the native protein was determined in this manner at approximately 110 kDA (see in this context FIG. 2, track 2). To study the enzymatic activity of the purified protein, a zymogram (track 3) was prepared. Track 1 contains the molecular weight standard.

In contrast, the molecular weight of the enzyme was determined by means of SDS-PAGE electrophoresis at 50 kDa (see in this context FIG. 1). The enzymatic activity was studied on the basis of the zymogram (track 6, crude extract and track 7, purified enzyme).

From the results it can be concluded that the purified amidase occurs preferentially as dimer of two identical subunits approximately 50 kDa in size.

In addition, the purified amidase was subjected to an endoproteinase Asp-N cleavage. Subsequently, from the approximately 110 kDa protein, a peptide having the mass 1482u can be isolated, which was sequenced by Edman degradation. This produced the SEQ ID No. 2.

### EXAMPLE 4

Determination of the Isoelectric Point

The isoelectric point was determined by means of isoelectric focusing using a Novex polyacrylamide gel (pH .3-10) and using a Novex electrophoresis chamber in accordance with the manufacturer's instructions (Novex, Invitrogen, Netherlands). The isoelectric point of the purified amidase was determined at pH 4.2.

### EXAMPLE 5

N-terminal Sequencing of the Purified Amidase

The purified amidase was transferred from the SDS-PAGE gel by electroblotting to a PVDF blotting membrane. The amino acid sequence of the N-terminal end of the resultant enzyme was determined by means of Edman degradation. This produced the SEQ ID No. 1.

A sequence comparison with other N-terminal ends of known amidases yielded no significant homologies.

In addition, the purified trimeric amidase was subjected to an endoproteinase Asp-N cleavage. Subsequently, from the approximately 150 kDa protein, a peptide having the mass 1482u can be isolated, which was sequenced using Edman degradation. This produced the SEQ ID No. 2.

### EXAMPLE 6

Enzyme Assays

General: Assay for Determining Hydrolytic Activity

To carry out the hydrolysis assay, where not mentioned otherwise, use was made of a 5 mM benzamide solution in 500 μl of a 60 mM K-Na phosphate buffer, pH 7.2. The enzymatic reaction proceeded for one hour at 70° C. with addition of 5 μg of the enzyme. The reaction was stopped by cooling the reaction solution on ice. The concentration of the products obtained enzymatically (benzoic acid and ammonia) was determined by means of HPLC and spectrometrically, using the ammonia kit Spectroquant 114752 (Merck, Germany). One unit (U) of amidase activity is defined as the amount of enzyme which catalyzes the formation of 1 μmol of benzoic acid per minute.

Assay for Determining the Acyltransferase Activity

The hydroxamic acid formation is determined by 10-minute incubation of the amidase at 70° C. in a mixture of 20 mM K-Na phosphate buffer, pH 7.2, 100 μl of a 50-100 mM amide and 0.7 M hydroxylamine solution (set to pH 7.0 with addition of 10 M NaOH). The hydroxylamine solution was prepared freshly for this. After 10 minutes' incubation at 50° C.-70° C., the reaction vessels were placed on ice and 1 ml of an acid iron chloride solution (0.1 M FeCl₃ in 50 ml of a 0.5 M HCl solution) was added. The resultant amount of hydroxamate was determined spectrometrically at a wavelength of 500 nm.

### EXAMPLE 6.1

Effect of Temperature and pH

The temperature dependence of the amidase activity was determined under standard conditions at pH 7.2. The reaction temperatures were varied between 30° C. and 90° C.

The results are shown graphically in FIG. 3, a temperature optimum was determined at approximately 70° C.

In addition, the temperature stability of the enzymes was studied between 50° C. and 80° C. over a relatively long period. The amidase isolated shows an excellent thermal stability at 60° C. (top curve, FIG. 5) and still a good stability at 70° C. (middle curve, FIG. 5). At a reaction temperature of 80° C., the enzymatic activity at pH 7.2 decreases rapidly, however (bottom curve, FIG. 5).

The pH optimum was carried out on the basis of hydrolysis of benzamide (5 mM) at 70° C. in a 50 mM buffer of sodium acetate (for pH 2-5) or sodium phosphate (for pH 5-13), the pH having been set successively up to pH 13 using 10 M NaOH.

The results are shown graphically in FIG. 4, a pH optimum at about 7 was determined.

### EXAMPLE 6.2

Effect of Metale and Inhibitors

To study the effect of certain reagents and ions on amidase activity, a solution containing 5 μg of amidase and the reagent or ion was preincubated for 1 h at 22° C. in 50 mM phosphate buffer. A 5 mM solution of benzamide in 50 mM phosphate buffer was then added until the end concentration of the reagent in the reaction batch was 1 mM. The reaction was performed for 1 h at 70° C. The catalytic activity was determined as described above.

The results are summarized in Table 2:

TABLE 2

| Reagent, 1 mM | Amidase activity in % |
|---|---|
| Cu²⁺ | 26 |
| Ni²⁺ | 42 |
| Zn²⁺ | 93 |
| Mg²⁺ | 90 |
| Ca²⁺ | 85 |
| Mn²⁺ | 98 |
| Fe²⁺ | 80 |
| Fe³⁺ | 96 |
| Ba²⁺ | 87 |
| Co²⁺ | 0 |
| EDTA | 100 |
| iodoacetamide | 90 |
| iodoacetate | 22 |
| SDS | 100 |
| Triton X-100 | 100 |
| DTT | 120 |

### EXAMPLE 6.3

Study of the Substrate Specificity of the Isolated Amidase

The substrate specificity of the amidase was tested at three different temperatures taking into account the substrate used in each case. For hydrolysis of amino acid amides, the reaction mixture was reacted for 3 h at 30° C., in the case of the aliphatic and cyclic amides 1 h at 50° C., and for aromatic amide substrates 1 h at 70° C. The enzymatic activity was then determined as described above. The control used was each batch without enzyme.

The substrates tested and the associated amidase activity are shown in Table 3:

TABLE 3

| | |
|---|---|
| | Specific activity |
| Substrate | (μmol min⁻¹ mg⁻¹) |

Aliphatic amides

| | |
|---|---|
| formamide | 9.2 |
| urea | 17.5 |
| acetamide | 15.7 |
| N-methylurea | — |
| acrylamide | 26.8 |
| DL-lactamide | 18.9 |
| malonamide | 7.4 |
| propionamide | 25.4 |
| fumaranamide | — |
| isobutyramide | 13.5 |
| methacrylamide | 23.5 |
| succinamide | 4.0 |
| pivalamide | 14.9 |
| adipamide | 12.0 |
| hexanoamide | 9.3 |
| cyclohexanoamide | 10.1 |

Aromatic amides

| | |
|---|---|
| sulfanylamide | — |
| 2-aminobenzamide | 3.4 |
| 4-aminobenzamide | 6.5 |
| benzamide | 19.4 |
| o-hydroxybenzamide | 7.4 |
| p-hydroxybenzamide | 12.1 |
| N-phenylurea | — |
| acetanilide | — |
| benzyl carbamate | — |
| o-tolylamide | — |
| m-tolylamide | 12.7 |
| p-tolylamide | 13.1 |
| 2-phenylpropionamide | 15.8 |
| 3-indolylacetamide | 7.56 |

Heterocycles

| | |
|---|---|
| pyrazinamide | 11.2 |
| nicotinamide | 11.6 |
| isonicotinamide | 7.1 |

Amino acid amides

| | |
|---|---|
| L-alaninamide | 10.5 |
| L-methioninamide | 14.4 |
| L-prolinamide | 13.0 |
| L-valinamide | 13.0 |
| L-leucinamide | 10.8 |
| L-tryptophanamide | 10.3 |
| 2-hydroxy-4-(methylthio)butyramide | 5.7 |
| L-tert-leucinamide | 0.08 |

### EXAMPLE 6.4

Study of the Enantioselectivity of the Isolated Amidase

For this, 10 μg of the purified amidase were incubated with a 5 mM racemic 2-phenylpropionamide solution for 3 h at 70° C., the end volume of the reaction batch being 500 μl. At 30-minute intervals, samples were taken from the reaction batch and analyzed by an HPLC measurement.

The product determination and quantification of the R-and S-stereoisomers of the 2-phenylpropionamide were determined by injecting defined amounts of the corresponding pure substances as standard. The enantiomeric excess (ee) was calculated on the basis of the peak area ratio obtained by the chirar HPLC measurement (ee^{p}=(S−R)/(S+R); ee^{p} (%)=(S−R)/(S+R) ×100; where p=product).

For the HPLC measurements, a chiral Chirobiotic T column (Astec, Whippany, USA) was used. The solvent used was ethanol and 20 mM potassium phosphate buffer, pH 7.2, having a volumetric ratio of 20:80. The elution was performed at room temperature using a flow rate of 0.8 ml min⁻¹. Detection was performed at a wavelength of 210 nm. The amfmonia concentration was studied using the phenol hypochloride method using the ammonia kit Spectroquant 114752 (Merck, Germany), the amount of ammonia released being determined spectrometrically using ammonium chloride as standard.

The results of the measurements are shown graphically in FIG. 7 (S-enantiomer top curve; R-enantiomer bottom curve). The purified amidase is highly S-selective with regard to 2-phenylpropionamide as substrate. After 60 minutes, the value of the enantiomeric excess ee^{p} was greater than 95, for a conversion of 50%.

1. A monomeric or multimeric amidase characterized in that the amidase contains an N-terminal sequence SEQ ID No. 1 or an N-terminal sequence having a homology of greater than 50% with SEQ ID No. 1.

2. A monomeric or multimeric amidase characterized in that the amidase contains a sequence SEQ ID No. 2 or a sequence having a homology of greater than 50% with SEQ ID No. 2.

3. The monomeric or multimeric amidase as claimed in claim 1, characterized in that the amidase contains an N-terminal sequence SEQ ID No. 1 and SEQ ID No. 2 or an N-terminal sequence having a homology of greater than 50% with SEQ ID No. 1 and a sequence having a homology of greater than 50% with SEQ ID No. 2.

4. The amidase as claimed in claim 1 having a molecular weight of the native monomeric enzyme between 47 and 53 kDa.

5. The amidase as claimed in claim 1, characterized in that the enzyme is obtainable from thermophilic bacteria.

6. The amidase as claimed in claim 1, characterized in that the enzyme is obtainable from Actinomycetes.

7. The amidase as claimed in claim 1, characterized in that the enzyme is obtainable from Pseudonocardia thermophila.

8. The amidase as claimed in claim 1, obtainable by a method comprising the method steps
a) centrifugation of the cell-free crude extract of a thermophilic bacterium at 10 000 to 20 000 rpm and subsequent addition of a 1 M salt solution,
b) chromatographic separation of the supernatant on a hydrophobic column using a reverse gradient of a salt solution from 1 M to 0 M,
c) ultrafiltration of the fraction showing amidase activity obtained from b) on a 10 kDa cut-off membrane,
d) ion-exchange chromatography of the protein fraction obtained from c) using a gradient from 0 M to 0.5 M of a salt solution, and
e) chromatography of the fraction showing amidase activity obtained from d) using a 100 to 200 mM salt solution and desalting the purified amidase fraction.

9. The amidase as claimed in claim 1, characterized in that the N-terminal end of the amidase containing the SEQ ID No. 1 or a sequence having a homology of greater than 50% with SEQ ID No. 1 is completely or partly deleted.

10. The amidase as claimed in claim 2, characterized in that the SEQ ID No. 2 or a sequence having a homology of greater than 50% with SEQ ID No. 2 of the amidase is completely or partly deleted.

11. The amidase as claimed in claim 1, characterized in that the enzyme is present as monomer or dimer, consisting of two monomeric amidase units.

12. The amidase as claimed in claim 1, characterized in that the enzyme has an amino acid sequence according to SEQ ID No. 3 or an amino acid sequence having a homology of at least 50% therewith.

13. A nucleic acid coding for an inventive amidase as claimed in claim 1, characterized in that the nucleic acid has a sequence according to SEQ ID No. 4 or a nucleotide sequence having a homology of greater than 60% therewith.

14. (canceled)

15. (canceled)

16. (canceled)

17. (canceled)

18. A method for the enzymatically catalyzed hydrolysis of amides, characterized in that the reaction is catalyzed by an amidase as claimed in claim 1.

19. The method as claimed in claim 18, characterized in that the reaction is carried out at a temperature between 30° C. and 85° C.

20. The method as claimed in claim 19, characterized in that the reaction is carried out at a temperature between 50° C. and 75° C.

21. The method as claimed in claim 18, characterized in that the reaction proceeds at a pH between 3.5 and 11.5.

22. A method of hydrolysis of amides or of acylation comprising contacting the amidase as claimed in claim 1 with an amide.

23. The method as claimed in claim 22 wherein the amides subject to hydrolysis are selected from the group consisting of aliphatic amides, aromatic amides, cyclic amides, heterocyclic amides and amino acid amides.

24. The method as claimed in claim 23 wherein the hydrolysis is enantioselective hydrolysis.

25. The method as claimed in claim 24 wherein S-stereoisomeric acids are produced.
